(19) United States
(12) Patent Application Publication
(10) Pub. No.: US 2006/0116681 A1
(43) Pub. Date: Jun. 1, 2006

(54) Surgical plate with transition zone capability

(76) Inventor: Jeffrey K. Bert, Coos Bay, OR (US)

Correspondence Address: CHERNOFF, VILHAUER, MCCLUNG & STENZEL, 1600 ODS TOWER, 601 SW SECOND AVENUE, PORTLAND, OR, 97204-3157, US

(21) Appl. No.: 11/000,621

(22) Filed: Nov. 30, 2004

(51) Int. Cl. A61F 2/30

(52) U.S. Cl. 606/69

(57) ABSTRACT

A surgical plate system including a surgical plate affixable to bone or other rigid tissue and a method for use of such a system. A connector body protruding from a secondary plate is matable in a receiver defined in a primary plate.

### BACKGROUND OF THE INVENTION

The present invention relates to surgical plates used to join two or more areas of bone or other tissue, and particularly to a method and apparatus for placing an additional plate immediately adjacent to or overlapping an area to which a plate was previously affixed.

Surgeons use surgical plates to immobilize a fractured bone to enable healing. Conventional surgical plates are integrally formed as rigid plates, and are secured to bone or other tissue by means of fasteners such as screws. A plate is placed approximately transversely to the fracture. Each end area of the plate is attached to bone, and the plate spans the fracture, so that fasteners are placed on either side of the fracture, allowing the plate to pull the fractured bone together and enable healing.

Plates are also used to fuse vertebrae, which is often necessary to relieve debilitating pain or correct a deformity in the spine. To perform a typical spinal fusion, the disk between the vertebrae which are to be fused is removed and a bone graft is inserted in its place. A plate is then attached to the vertebrae, spanning the graft, and the plate immobilizes the vertebrae adjacent to the graft until the fusion is complete. Fasteners are used to attach the plate to both vertebrae, so that the plate spans the bone graft and immobilizes the area of the spine which is to be fused. A conventional plate may be generally rectangular and may have holes in its corners through which fasteners such as screws extend into the bone against which the plate is placed.

Often, once a spinal fusion or fracture repair is made, especially in osteoporotic individuals, the patient experiences a complication called transition zone syndrome. Transition zone syndrome is a premature degeneration of a section of bone adjacent to a fusion or plate placement. The causes of transition zone syndrome are not well understood. It is hypothesized that increased stresses transferred to the adjacent segment of bone by the previous surgical procedure precipitate the premature degeneration. If transition zone syndrome occurs, an additional bone fracture or the weakening, damage, or rupture of a disk can occur immediately adjacent to the previous plate, requiring an additional repair immediately adjacent to the old plate. Unfortunately, such a repair is very difficult to perform using conventional methods. The old plate over time becomes embedded in tissue, and significant displacement of such tissue is required to gain access to the old plate, which conventionally must be unscrewed and pried off of the bone. This is difficult and potentially dangerous. Accordingly, when faced with the problem of a new injury adjacent to an old plate, surgeons may be unable to use conventional fasteners to affix the new plate to the bone. Surgeons may tie the new plate to the bone, but this is less effective than use of a screw or conventional fastener. Moreover, plates are intended to function as tension bands. Tension is force tending to separate the bones or fractured parts of bones longitudinally to the plate. Compression is force tending to compress the bones or fractured parts of bones. Plates are intended to strengthen or immobilize a bone or other tissue construct by maintaining tension rather than resisting compression. They are generally attached to the convex side of a curved long bone for example, and pull the fractured parts of the bone together. Therefore, a plate should be able to withstand axial tension, or force tending to separate bones held together by a plate longitudinal to the plate. A plate that is tied to the bone instead of screwed to the bone may not effectively withstand axial tension.

What is desired, then, is to be able to attach a secondary plate to the area adjacent to a pre-existing plate in such a way as to preserve the secondary plate's function as a tension band without removing the pre-existing plate.

### SUMMARY OF THE INVENTION

In accordance with a first aspect of the present invention, a plate system attachable to body tissue for interconnecting two adjacent areas of body tissue is provided in which a first plate having side margins and ends, said plate defining a receiver, is installed to support healing bone tissue. A secondary plate including a connector body shaped to matingly fit into the receiver is provided. The secondary plate may be added at a time subsequent to placement of the first plate and is attached to the first plate by the connector body and to the bone by fasteners such as screws.

Another aspect of the invention is a method of surgically providing a support mechanism for adjacent body structures by applying a plate system to body tissue, wherein a first plate defines a receiver, matingly connectable with a connector body found on a second plate.

In one embodiment of the method, the first plate is attached to bone by fasteners, then the secondary plate is attached to the first plate, by mating the connector body with the receiver, and is attached to the bone by fasteners.

The foregoing and other objectives, features, and advantages of the invention will be more readily understood upon consideration of the following detailed description of the invention taken in conjunction with the accompanying drawings.

### BRIEF DESCRIPTION OF THE SEVERAL VIEWS OF THE DRAWINGS

FIG. 1 is a simplified front view of a section of spine to which is affixed a surgical plate embodying an aspect of the invention.

FIG. 1A is a section view taken on line 1A-1A in FIG. 1.

FIG. 2 is a front view of a section of spine to which is affixed a surgical plate such as that shown in FIG. 1, together with an isometric view of a special punch, and showing an area internal to the receiver of the plate cleaned of tissue by use of the punch.

FIG. 3 is a front view of a section of spine to which are affixed a plate as shown in FIG. I and a secondary plate.

FIG. 3A is a sectional view taken along lines 3A-3A in FIG. 3.

FIG. 4 is a top view of an alternate embodiment of the plate system disclosed herein.

FIG. 5 is a top view of a second alternate embodiment of the plate system disclosed herein.

FIG. 6 is a top view of a third alternate embodiment of the plate system disclosed herein.

FIG. 7 is a top view of a long bone fracture to which are affixed a plate as shown in FIG. I and a secondary plate.

### DETAILED DESCRIPTION OF PREFERRED EMBODIMENTS

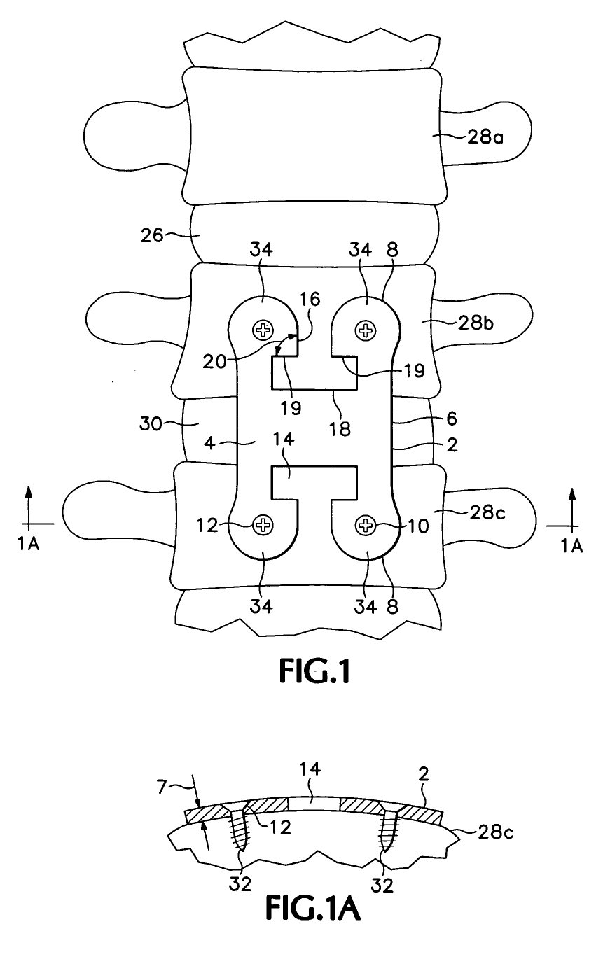

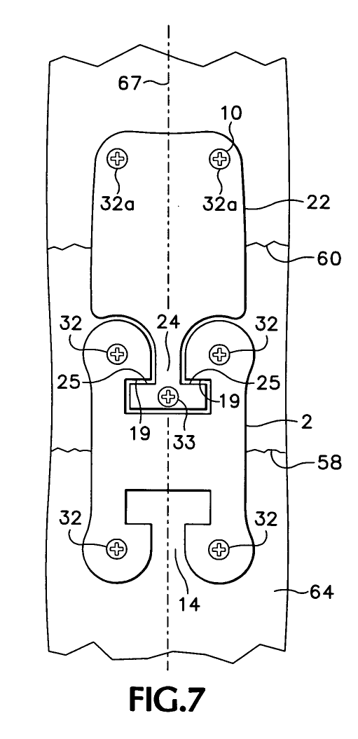

Referring now to FIGS. 1-1A of the drawings which form a part of the disclosure herein, a surgical plate 2 is integrally formed from a rigid material and is attachable to rigid body tissue such as bone. The plate 2 may be attached to vertebrae or a long bone 64 such as a femur (see FIG. 7). The plate 2 has a body 4 including side margins 6 and end 8. The plate 2 is manufactured using techniques known in the art and may be made of biocompatible material, such as titanium, titanium alloy, or stainless steel. The plate 2 should be relatively light in weight but should be strong enough to withstand expected amounts of tension in use. It should be relatively thin and generally planar or may be slightly curved as shown in FIG. 1A, to conform to the shape of bone to which it is intended to be attached. The plate 2 may have a thickness 7 of 2.5 mm for example, although the thickness 7 is determined depending on where it is designed to be placed. It has receptacles 12 to accommodate fasteners 10 such as screws, nails, or tacks and can include a conventional locking mechanism (not shown) to immobilize them once they are installed. The receptacles 12 can preferably accommodate up to 15 degrees of variation in fastener angle in order to allow the vertebrae being fused together to subside, or move closer to one another, as healing occurs.

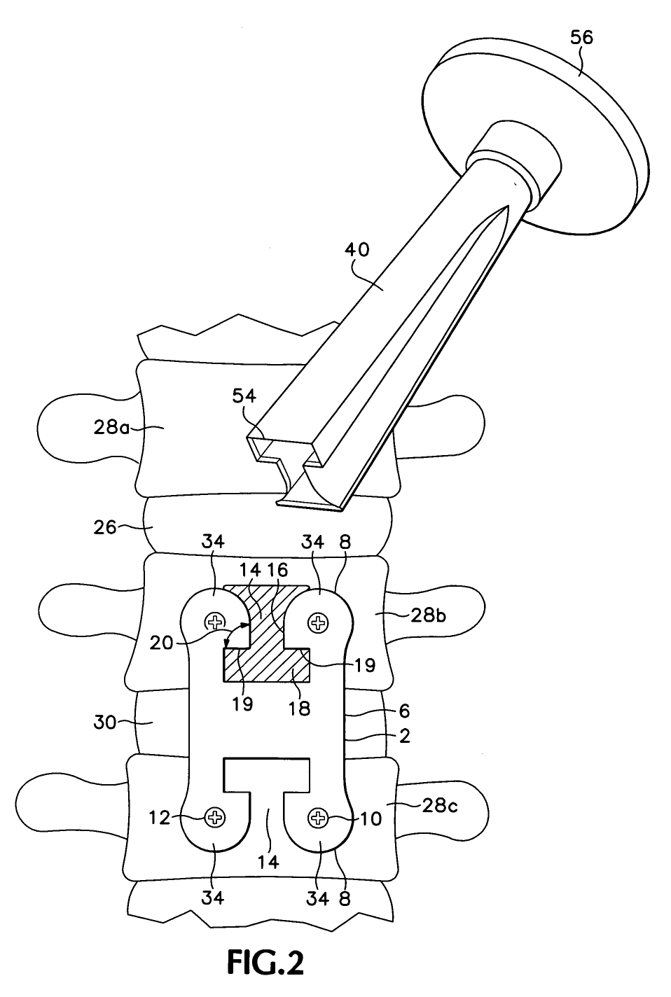

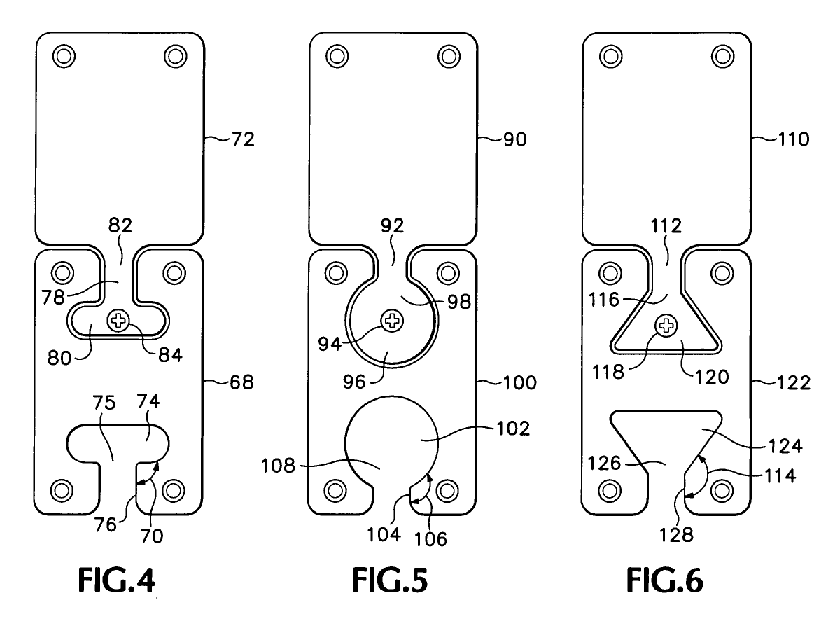

The plate 2 includes a receiver 14 in one or both ends 8. The receiver 14 is shaped such that a relatively narrow channel 16 extends to a larger bay 18. The difference in width between the channel 16 and bay 18 is defined by a shoulder angle 20 (see FIG. 2) between the sides of the channel 16 and surfaces 19 defining the bay 18. The shoulder angle 20 should be less than 145° and must be less than 180°, and the surfaces 19 are preferably perpendicular to the general plane of the plate 2. The shoulder angle 20, relatively narrow channel 16, and larger bay 18 allow a secondary plate 22 with a connector body 24 sized and shaped to fit matingly with the receiver 14 to lock in place so that the construct resists tension in a direction axial to the plate along line 67 (see FIG. 7), so that the plates cannot be pulled apart once the plates are connected. The specific shape of the receiver may vary. It may be characterized by a narrow channel 104 and a round bay 102 (FIG. 5), a triangular bay 124 (FIG. 6), a bar-shaped bay such that the channel and bay form a T-shape 74 (FIG. 4), or other variations.

When a surgical procedure is performed to create spinal fusion, such as a fusion of two or more cervical vertebrae, the disk 26 between two adjacent vertebrae 28b, 28c is removed, a bone graft 30 is inserted, and a plate 2 is affixed to the vertebrae such that it spans the graft 30 and immobilizes the vertebrae relative to the graft. The immobilized vertebrae 28b, 28c, then fuse together with the graft 30, as a result of biological action, and tissue will eventually grow tightly adjacent the ends 8 and side margins 6. The plate 2 is affixed to the vertebrae 28b, 28c in the same manner in which conventional surgical plates are implanted, using fasteners 10 placed preferably in each of the corners 34 defined generally by the intersection of the side margins 6 and ends 8.

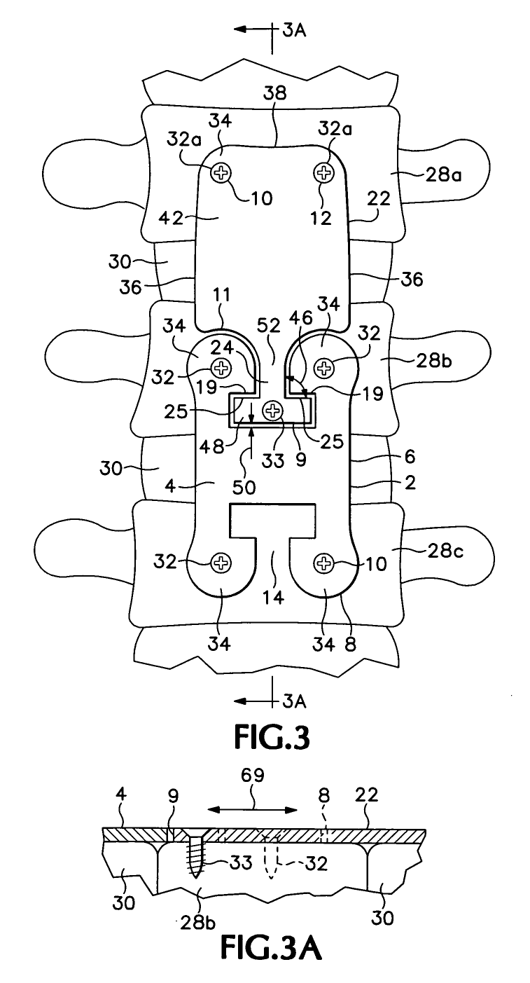

After the procedure is completed, the patient may experience a subsequent fracture, rupture, or deformity of a disk adjacent to the fused vertebrae. This subsequent injury may require further repair employing a surgical plate immediately adjacent to the first plate. In this event, the injured disk is removed, and the first plate is prepared for attachment of the secondary plate. A punch 40 specially shaped to fit matingly within the receiver is used to remove tissue which has subsequently formed within the receiver 14, as shown in FIG. 2. The punch 40 has a handle 56 which permits the surgeon to apply pressure manually or by use of a mallet to chisel away any tissue accumulated within the receiver 14. The punch has sharp edges 54 which facilitate the removal of tissue. Other tools commonly used for scraping or chiseling may also be used to remove accumulated tissue. Once sufficient tissue has been removed, a secondary plate 22 is installed, as shown in FIG. 3. The secondary plate 22 includes receptacles 12 for fasteners 10. The secondary plate has a connector body 24 which is shaped to fit matingly within the receiver 14 of the plate 2, the connector body 24 including a narrow neck portion 52 corresponding with the channel 16 of the receiver 14 and wider head 48 corresponding with the bay 18 of the receiver in shape and size. The connector body 24 is inserted into the receiver 14 and fasteners 10 are used to fasten the secondary plate to the vertebrae on either side of the new fusion. Preferably at least one fastener such as a screw 33 is placed through the connector body 24, fastening the secondary plate 22 to one of the fused vertebrae 28b to which the original plate 2 is also attached so that the plates are aligned to be coplanar as shown in section view in FIG. 3A, so that the surfaces 19 of the bay 18 facing away from the end 8 are aligned with and face toward the surface 25. Further fasteners 32a are used to attach the main body 42 of the secondary plate 22 to the vertebra 28a on the other side of the graft 30 so that the secondary plate spans the new vertebra to be fused. Once the connector body 24 is mated with the receiver 14, the plate system can withstand axial tension because the plates are interconnected and cannot be separated by force in an axial direction, indicated by the arrow 69 in FIG. 3A. The shoulder angle 20 of the receiver channel 16 and bay 18 corresponds with the shoulder angle of the neck 52 and head 48 of the connector body 24 so that the two plates interlock and cannot be pulled apart. Thus the interconnection between the mated plates can withstand axial tension. As shown in FIGS. 3 and 3A the fastener 33 maintains the connector body 24 in a coplanar relationship with the receiver 14. The first plate 2 is anchored by corner fasteners 32. When the connector body and receiver are coplanar, tension forces cause the corresponding surfaces 19 on the sides of the bay 18 which face away from the end 8 of the first plate to press against opposed corresponding surfaces 25 of the connector body 24, allowing the plate system to resist tension.

The fit between the connector body 24 and the receiver 14 is relatively tight, with approximately one-half to one millimeter of gap 50 between the two components. In this way, the fit is tight enough to permit the plate construct to function as a tension band, but the fit is loose enough to allow the placement of the transition plate notwithstanding the accumulation of tissue in the receiver 14 and possible incomplete removal with the punch 40. The receiver 14 and connector body 24 can take a variety of shapes. FIGS. 4, 5 and 6 show alternative shapes.

FIG. 4 shows an alternate embodiment of the plate system in which the plate 68 has a rounded T-shaped receiver 75 including a narrow channel 76 and the secondary plate 72 has a rounded T-shaped connector body 78 including a bar shaped head 80. The shoulder angle 70 defined by the intersection between the channel 76 and the bay 74 is approximately 90 degrees. A fastener 84 is placed in the connector body 78 to hold it in generally coplanar alignment with the receiver 75 in the plate 72.

FIG. 5 shows yet another alternate embodiment of the plate system in which the plate 100 has a round receiver 108 including a round bay 102 and a narrow channel 104, and the secondary plate 90 has a connector body 98 with a round head 96 with a narrow neck 92. The shoulder angle 106 defined by the intersection between the channel 104 and the head 102 is between 100 degrees and 120 degrees. A fastener 94 is placed in the connector body 96, to hold it in generally coplanar alignment with the receiver 108 in the plate 100.

FIG. 6 shows another alternate embodiment of the plate system in which the plate 122 has a receiver 126 with a narrow channel 128 and a triangular shaped bay 124, and the secondary plate 110 has a connector body 116 with a triangular head 120 and narrow neck 112. The shoulder angle 114 defined by the intersection between the channel 128 and the bay 124 is between 100 degrees and 170 degrees. A fastener 118 is placed in the connector body 116 to hold it in generally coplanar alignment with the receiver 126 in the plate 122.

FIG. 7 shows a long bone 64 with a fracture 58 spanned by a plate 2. Subsequent to installation of the surgical plate 2 the patient has developed transition zone syndrome, a secondary transition zone fracture 60 has occurred next to the pre-existing plate 2. To repair the secondary, transition zone fracture 60, a secondary plate 22 has been installed and spans the second fracture 60. The punch 40 is used to remove tissue from the area of the receiver 14 (see FIG. 2). The secondary plate 22 is installed by inserting the connector body 24 into the receiver 14 and attaching it to the bone 64 with fasteners 10. The connector body 24 includes at least one fastener 33 to hold it in generally coplanar alignment with the receiver 14 in the plate 2. Because of the fastener 33 in the connector body, fasteners are placed in either side of the new fracture, better enabling the plate system to perform its immobilization function as a tension band.

The plate system can be used to immobilize any bone or rigid tissue found in the human or any mammalian body, where conventional plates may be used.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

What is claimed is:

1. A plate system attachable to rigid body tissue for interconnecting two adjacent areas of rigid body tissue, comprising:
(a) a first plate having a body having an end and a pair of opposite side margins, said end interconnecting said side margins, and said body defining a receiver proximate to said end;
(b) a second plate having a main body and an end interconnecting a pair of opposite side margins;
(c) said end of said second plate defining a connector body shaped to fit matingly within said receiver; and
(d) said plate system having a plurality of fasteners associated with each of said plates for fastening each of said plates to respective ones of said areas of body tissue so as to interconnect said areas of body tissue with one another, at least one of said fasteners being associated with said connector body.

2. The plate system of claim 1 wherein said fasteners are screws.

3. The plate system of claim 1, wherein said connector body includes a relatively large head and a relatively narrow neck interconnecting said head with said main body of said second plate.

4. The plate system of claim 3, wherein said head is approximately triangular.

5. The plate system of claim 1, wherein said receiver is approximately T shaped.

6. The plate system of claim 1 wherein the plate system is capable of withstanding tension between said first and second plates after said plates are fastened to said areas of body tissue with said connector body fitted in said receiver.

7. A method of interconnecting at least two adjacent boney areas, comprising:
(a) providing a plate system comprising a first plate and a second plate;
(i) said first plate having at least one end interconnecting two side margins, said end defining a receiver;
(ii) said second plate having at least one end interconnecting a pair of opposite side margins, said end including a connector body;
(iii) said connector body fitting matingly within said receiver; and
(iv) said plate system including a plurality of fasteners associated with each of said plates for fastening each of said plates to said boney areas so as to interconnect said boney areas;
(b) attaching said first and second plates to each other by inserting said connector body into said receiver; and
(c) fastening said plates to said boney areas with at least some of said plurality of fasteners, at least one said fastener being located in said connector body.

8. The method of claim 7 wherein said fasteners are screws.

9. The method of claim 7, wherein said connector body includes a relatively large head and a relatively narrow neck interconnecting said head with said second plate.

10. The method of claim 9, wherein said head is approximately triangular in shape.

11. The method of claim 7, wherein said receiver is approximately T shaped.

12. The method of claim 7, wherein the plate system is capable of withstanding tension between said first and second plates after said plates are fastened to said areas of body tissue with said connector body fitted in said receiver.

13. A method of connecting two boney areas comprising:
(a) providing a plate having an end interconnecting a pair of opposite side margins, said plate defining a receiver communicating with said end, said receiver having a large bay and a narrower channel extending between said bay and said end of said plate, and said plate having one or more receptacles for the placement of fasteners; and
(b) fastening at least one of said fasteners through a receptacle in said plate to each said boney area so as to interconnect said boney areas with said plate.

14. The method of claim 13 wherein said receiver is approximately T shaped.

15. The method of claim 13 wherein said receiver has a narrow channel and a wider bay.

16. A surgical plate shaped to connect with a corresponding second plate, said surgical plate comprising a body having at least one end interconnecting two side margins, said end defining a receiver.

17. A surgical plate having a body, a pair of opposite side margins, and an end interconnecting the side margins, said body defining a receiver proximate said end, said receiver including a surface facing inwardly and generally away from said end and oriented so as to be capable of sustaining a load directed away from said end.
